(19) United States
(12) Patent Application Publication
(10) Pub. No.: US 2006/0010652 A1
(43) Pub. Date: Jan. 19, 2006

(54) Self-retained anti-microbial door knob cover

(76) Inventors: Martin Kellaher, Guilford, CT (US); Harry M. Brady, Guilford, CT (US)

Correspondence Address: ALIX YALE & RISTAS LLP, 750 MAIN STREET, SUITE 1400, HARTFORD, CT, 06103, US

(21) Appl. No.: 11/179,945

(22) Filed: Jul. 12, 2005

Related U.S. Application Data
(60) Provisional application No. 60/587,411, filed on Jul. 13, 2004.

(51) Int. Cl. A45C 13/26; A61F 2/00

(52) U.S. Cl. 16/413; 424/423

(57) ABSTRACT

A replaceable cover for a handle in public use, comprising a soft, molded elastomeric membrane of thickness in the range 0.01 to 0.25 inch impregnated with an antimicrobial agent, that is sufficiently flexible to be stretched over and snugly form fit and self-retained on the handle without adhesive. The membrane can be a thin, hollow shell that is open at one end and has three mutually orthogonal axes on respective mutually orthogonal planes. The membrane forms a surface that is traceable as a substantially continuously curved line in each of these planes from a fixed center within the shell, spanning at least about 270 degrees about the center. The invention is also directed to the combination of a handle having a body and a rigidly projecting rotatable stem, with a replaceable, stretchable, elastomeric cover impregnated with an antimicrobial agent that is snugly form fit onto the body over at least about 65% and preferably at least 75% of the surface area of the body.

### RELATED APPLICATION

This is the regular application of U.S. Provisional Application No. 60/587,411 filed Jul. 13, 2004, from which priority is claimed under 35 U.S.C. Sec. 119(e) and the entire disclosure of which is incorporated by reference.

### BACKGROUND OF THE INVENTION

The present invention is directed broadly to a replaceable elastomeric soft cover for surfaces subject to handling by many humans in everyday activities, such as knobs, levers, latches, and the like that may be found, for example, in connection with certain types of toilet bowl flushers, towel dispensers, and in particular to a door knob cover.

A number of U.S. patents such as U.S. Pat. Nos. 2,527,955, 4,832,942, 5,882,667 and 6,546,594 describe flexible or rigid disinfectant or anti-microbial covers or sleeves for push plates, handles, knobs and the like, wherein an adhesive or other retention means is required for bodies having a complex shape.

U.S. Pat. No. 6,520,281 describes an elastomeric antimicrobial cover for a stethoscope head having a complex shape. U.S. Pat. Nos. 4,975,826, 5,937,877, 5,599,097, 5,355,292 and 4,777,574 also relate to prophylactic coverings for equipment used during the delivery of medical treatment. However, a stethoscope and other medical devices or equipment are not intended for public use and thus are not handled by many humans in public.

### SUMMARY OF THE INVENTION

The present invention is directed to a replaceable cover for a handle in public use, comprising a soft, molded elastomeric membrane of thickness in the range 0.01 to 0.25 inch impregnated with an antimicrobial agent, that is sufficiently flexible to be stretched over and snugly form fit and self-retained on the handle without adhesive.

The membrane can be a thin, hollow shell that is open at one end and has three mutually orthogonal axes on respective mutually orthogonal planes. The shell forms a surface that is traceable as a substantially continuously curved line in each of these planes from a fixed center within the shell, spanning at least about 270 degrees about the center.

The invention is also directed to the combination of handle having a body and a rigidly projecting rotatable stem, with a replaceable, stretchable, elastomeric cover impregnated with an antimicrobial agent that is snugly form fit onto the body over at least about 65% and preferably at least 75% of the surface area of the body.

The preferred handle is for operating a publicly accessible door, wherein the cover is a soft, molded elastomeric membrane of thickness in the range 0.01 to 0.25 inch that is stretched over and snugly form fit and self-retained on the handle without adhesive.

A significant feature of the preferred embodiment is the form fitting of the anti-microbial elastomeric cover, which can be stretched over and self-retained on a complex shaped body, without adhesive.

In the present context, complex shaped body means a body having three significant dimensions, each of which is defined by a substantially continuously curved surface traceable from a fixed center within the body, e.g., the curve may be drawn with varying radii from a center and spans at least about 270 degrees about the center. Both the handle and the cover span a solid angle of at least 270 degrees. Typical bodies of this type include spheres, ellipsoids, and solid sections (truncations) thereof. Also, public use should be understood in contrast from, e.g., use of medical equipment or certain personal paraphernalia such as toothbrushes, writing instruments, and the like. The invention is intended for use in environments where antimicrobial surfaces are desirable but absolute sterility is not normally required, monitored, or maintained, and thus includes doors and the like in private homes as well as in truly public facilities.

### BRIEF DESCRIPTION OF THE DRAWING

The invention will be exemplified with reference to the accompanying drawing, in which:

FIG. 1 is a side view of a door knob with self-retained cover according to one embodiment of the invention;

FIG. 2 is a perspective view of the door knob and cover of FIG. 1;

FIG. 3 is a schematic representation of the cover of FIG. 1 after placement over the door knob, and labeled to define various significant characteristics;

FIG. 4 is a schematic sectional view on plane A-C of FIG. 3;

FIG. 5 is a schematic sectional view on plane A-B of FIG. 3; and

FIG. 6 is a schematic sectional view on plane B-C of FIG. 3.

### DESCRIPTION OF THE PREFERRED EMBODIMENT

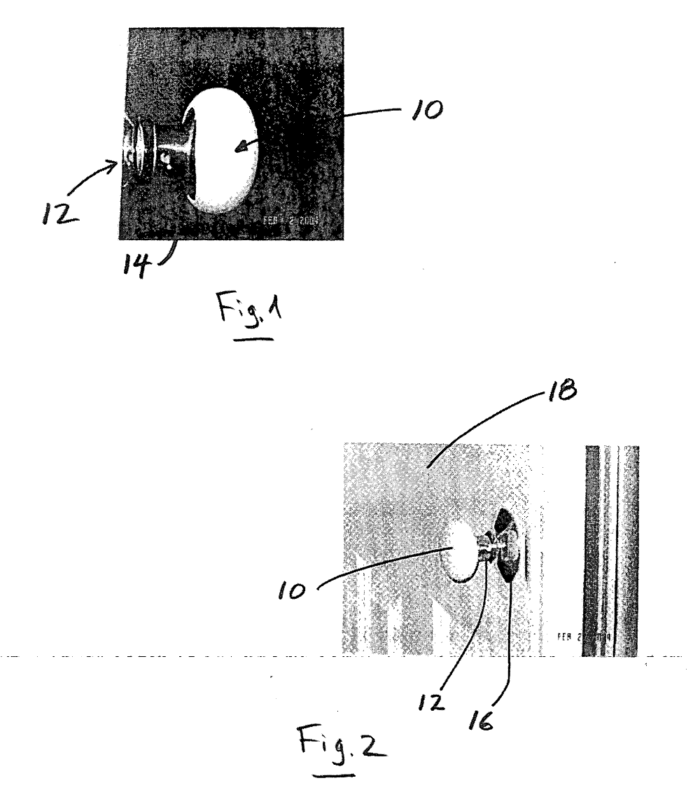

The preferred embodiment will be described with reference to FIGS. 1-6, but it should be understood that variations could also fall within the scope of the invention. The Figures show the inventive cover 10 fit snugly over a conventional door knob 12, comprising a substantially ellipsoid body 14 and rigid stem or spindle for operating a latch or the like with associated flange 16 by which the door knob is attached to a door 18. The elastomeric cover 10 consists of a flexible, homogeneous membrane that has the overall shape of a shower cap, but the entire cover, not just the open portion, can be stretched over the door knob body 14 and then released to produce the snug fit, without the use of either adhesives or a separate resilient ring or the like. Despite the snug fit, the cover can be easily removed and replaced after it has shown signs of wear or become discolored from extensive handling.

In general, the elastomeric material has a durometer measurement in the Shore A classification, and ranges in thickness from about 0.01 to about 0.25 inch, depending on end use. The material is impregnated with anti-microbial agent during or immediately after injection molding into the desired shape, but retains a softness that provides a comfortable, secure texture when grasped. The tactile sensation is quite pleasing due to the combination of softness and snug fit on the body portion 14 of the door knob. Any of a variety of anti-microbial agents can be used, such as silver ions, or Triclosan®, which maintains its ability to sanitize and prevent the spread of harmful bacteria and other pathogens on the cover as different individuals successively touch or operate the door knob.

Preferably, the cover is produced by an injection molding process wherein the elastomeric material with anti-microbial agent is heated then injected into a mold cavity. In the mold cavity the material is cooled and formed into a complex-shaped membrane. The mold opens and the membrane is ejected, ready for use.

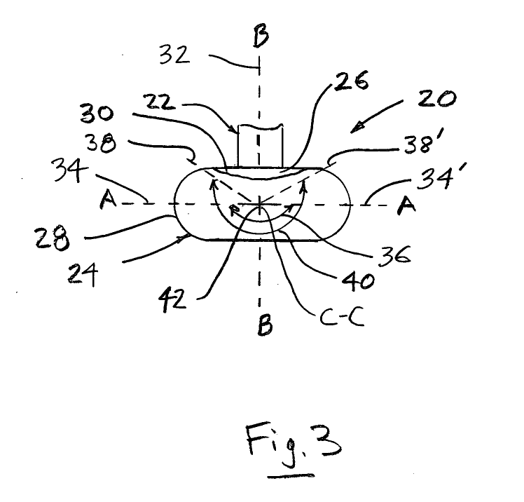

FIG. 3 is a diagrammatic representation of a door knob assembly 20 having a stem 22 and protected knob 24 consisting of body 26 and elastomeric cover 28. The cover has an opening defined by circular edge 30, which is flexible enough to be expanded over the body 26 as the entire cover 28 stretches. The stem 22 defines a rotation axis 32, and the ellipsoid body defines a major axis 34 that is orthogonal to and intersects with the rotation axis, at the center of the body. The body has three significant dimensions, (1) along major axis 34-34′, in the plane AC defined by A-A and C-C, (2) along the rotation axis 32, in the plane BC defined by axes B-B and C-C, and (3) along an axis 42 into and out of the plane AB, of the drawing, in the plane defined by A-A and B-B. It can be appreciated that a view of the assembly 20 taken from the left or right along the axis 34, 34′ would appear the same as FIG. 3, except that the present axis 42 seen as a point would reverse roles with the axis 34, 34′ currently seen as a line. Typical bodies for door knobs are substantially spherical or substantially ellipsoidal, and many other handles comprising complex shaped bodies can be considered as having an outer surface that is continuously convexly curved in the directions of each of the three axes.

Even if such bodies are not continuously convexly curved, for example having regions that are substantially flat, they can be considered as smoothly curved. In this context, smoothly curved means that a ray drawn from the center and rotated through 360 degrees on each of the three mutually orthogonal planes defined by the three axes would not make an abrupt change in direction, e.g., no region of the surface along such a plane forms an incremental angle of more than about 75 degrees per degree change in ray position.

FIG. 3 shows that the cover continuously encapsulates the body over a solid angle of at least about 200 degrees, preferably about 240-300 degrees. The angle 36 defined between opposite segments 34, 34′ of the major axis is 180 degrees, and the angle 40 indicated by lines 38, 38′ nearly represents the extent of the cover and is well over 200 degrees. That portion of the body 26 that is not covered provides the region associated with the connection to the stem 22.

The cover 28 before application onto the body has substantially the same shape as the surface of the body within the solid angle range of 200-300 degrees. Thus, the cover apart from the knob forms a thin, flexible, homogeneous, continuously curved membrane shell having a center and spanning a solid angle of at least about 200 degrees, preferably 240-300 degrees about the center, thereby defining a substantially circular opening.

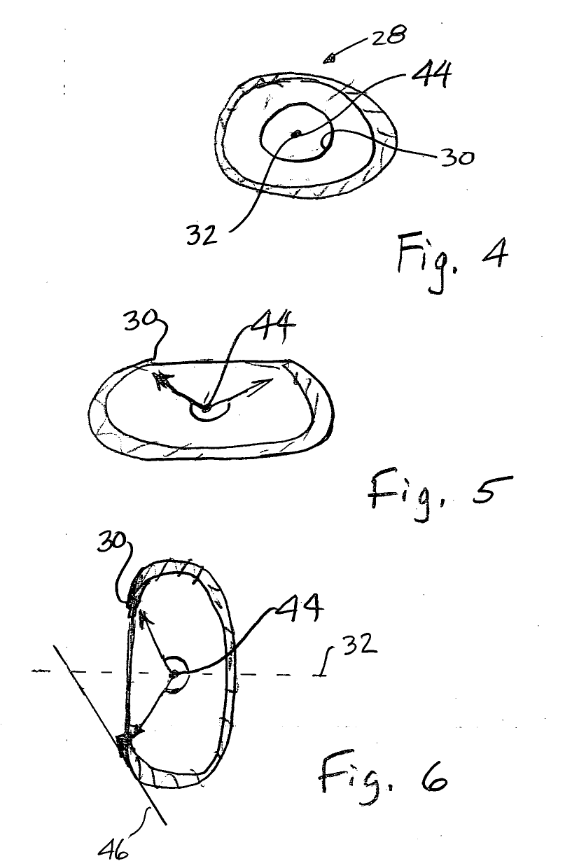

FIGS. 4-6 are schematic section views of the cover 28 of FIG. 3 in plane A-C looking along axis 32 toward the opening 33; in plane A-B looking along axis 42 into the paper, and in plane B-C looking along axis 34 toward the right. Various rays are drawn which together span an included angle indicated at 40 in FIG. 3. FIG. 4 shows an included angle of 360 degrees. FIGS. 5 and 6 show a substantially continuous curvature from the center 44, over at least 200 but less than 360 degrees. It can be appreciated that the paths traced at the surface in each of these planes of FIGS. 5 and 6 is continuously convexly curved if the membrane is a true spherical or elliptical shell, whereas the surface shown in FIGS. 5 and 6 may have a flat portion at the apex and therefore can be considered smoothly curved, even if they are not continuously convexly curved.

When humans grasp a door knob they often place the main body against their palm and wrap the fingers over the body and curl them against the backside of the body. For this reason, the cover should snugly envelope as much of the backside of the body as possible, both for anti-microbial purposes and for consistency in tactile sensation between the palm and the fingers. Other covered body shapes having a projecting rigid, rotatable stem are also within the scope of the invention, especially where the body is two or three way symmetric about a center through which the stem axis passes. In general, the cover will envelope at least 65%, and preferably at least 75% of the surface area of the body, and be snug throughout.

It can be appreciated that if the surface curvature spans more than 180 degrees and is continuously convexly curved (at least at the periphery), the surface will, in essence, curve back on itself. Such curving back can also be achieved, as shown in FIGS. 3, 5, and 6, even if a portion of that surface is flat. In this context, “curve back” means that the surface spans at least 180 degrees and, at the termination of such surface, has a tangent 46, which intersects the centerline 32 of the body or the cover. This centerline 32 as shown in FIG. 3, is also the rotation axis for the handle.

Installation can be facilitated by wiping the target surface with a pad or the like that is saturated with alcohol, for cleaning the surface. Any residual alcohol remaining on the surface can help the cover slide onto and stretch over the surface of the handle, and thereafter the alcohol quickly evaporates through the cover. There is no adhesive effect, in that the cover can be removed by stretching and peeling back the cover over the knob or handle.

1. A replaceable cover for a handle in public use, comprising a soft, molded elastomeric membrane of thickness in the range 0.01 to 0.25 inch impregnated with an antimicrobial agent, that is sufficiently flexible to be stretched over and snugly form fit and self-retained on said handle without adhesive.

2. The cover of claim 1 wherein the membrane is a thin hollow shell that is open at one end and has three mutually orthogonal axes on respective mutually orthogonal planes, said shell forming a surface that is traceable as a smoothly curved line in each of said planes from a fixed center within the shell and spanning at least about 270 degrees about the center.

3. The cover of claim 1, wherein the membrane substantially defines the surface of a truncated hollow sphere or ellipsoid.

4. The cover of claim 2, wherein the shell follows a substantially continuously convex curvature in the directions of each of the three axes.

5. A handle having a body and a rigidly projecting rotatable stem, comprising a replaceable, stretchable, elastomeric cover impregnated with an antimicrobial agent and snugly form fit onto the body over at least about 65% of the surface area of the body.

6. The handle of claim 5, wherein the handle is a publicly accessible door knob.

7. The handle of claim 5, wherein the cover is a soft, molded elastomeric membrane of thickness in the range 0.01 to 0.25 inch that is stretched over and snugly form fit and self-retained on said handle without adhesive.

8. The handle of claim 7, wherein the handle is a spheroidal or elliptical knob and the cover substantially defines the surface of a truncated hollow sphere or ellipsoid.

9. The handle of claim 8, wherein the cover follows a continuously convex curvature in the directions of each of three mutually perpendicular axes.

10. The handle of claim 6, wherein the body spans a solid angle of at least about 270 degrees and the cover continuously encapsulates the body over a solid angle of at least about 200 degrees.

11. The handle of claim 6, wherein the cover is form fit over at least 75% of the surface of the body.

12. A replaceable cover for a handle in public use, consisting of a homogeneous, molded elastomeric Shore A membrane of thickness in the range 0.01 to 0.25 inch impregnated with an antimicrobial agent, that has a complex shape and is sufficiently flexible to be stretched over and snugly form fit and self-retained on a handle of complex shape, without adhesive.

13. The cover of claim 12, wherein the membrane has a thickness in the range of about 0.05 to about 0.15 inch.

14. The cover of claim 12, wherein the membrane substantially defines the surface of a truncated hollow sphere or ellipsoid.

15. The cover of claim 12, wherein the membrane is a thin hollow shell that is open at one end and has three mutually orthogonal axes on respective mutually orthogonal planes, said shell forming a surface that is traceable as a smoothly curved line in each of said planes from a fixed center within the shell and spanning at least about 270 degrees about the center.
